(19) United States
(12) Patent Application Publication
(10) Pub. No.: US 2015/0352303 A1
(43) Pub. Date: Dec. 10, 2015

(54) DEVICE HAVING MANUAL RESUSCITATION AND SUCTION CAPABILITIES

(71) Applicant: Grant Godwin, Oakboro, NC (US)

(72) Inventor: Grant Godwin, Oakboro, NC (US)

(21) Appl. No.: 14/719,869

(22) Filed: May 22, 2015

Related U.S. Application Data
(60) Provisional application No. 62/008,008, filed on Jun. 5, 2014.

(51) Int. Cl. A61M 16/00; A61M 16/20; A61M 16/04

(52) U.S. Cl. CPC A61M 16/0084; A61M 16/0463; A61M 16/208

(57) ABSTRACT

Disclosed is a resuscitator including a valve assembly having a check valve arranged within a valve body, the valve body defining a gas passageway therethrough, the valve body including a first inlet, a second inlet and a first outlet, wherein the second inlet and the first outlet are axially aligned, and the first inlet is axially perpendicular to both the second inlet and the first outlet, and wherein the valve is arranged within the valve body between the second inlet and the first outlet in axial alignment with the second inlet and the first outlet; a compressible bag in fluid communication with the first inlet; a suction device removably attached to the second inlet; and an endotracheal tube removably attached to the first outlet. The suction device includes a tube arranged to be advanced through the valve and into the endotracheal tube such that the endotracheal tube can be suctioned without having to detach the endotracheal tube from the valve body.

### CROSS-REFERENCE TO RELATED APPLICATION

This U.S. Non-Provisional Patent Application claims priority from U.S. Provisional Patent Application No. 62/008,008 filed on Jun. 5, 2014, which is incorporated herein by reference in its entirety.

### TECHNICAL FIELD

The present invention relates to the field of pulmonology, and more particularly, to devices having a dual capacity for manual resuscitation and suction (e.g., aspiration).

### BACKGROUND OF THE INVENTION

In critical care situations occurring either in the field or a hospital setting, manual resuscitation is often required to assist a patient with breathing. During these critical care situations, a tracheal tube (e.g., an endotracheal tube) may either be inserted into the patient's trachea or already present in the patient's trachea. In certain situations, for example, when a patient is coding, manual resuscitation is often needed in lieu of mechanical ventilation. For example, if a patient's tracheal tube is attached to the mechanical ventilator, the mechanical ventilator is detached and a bag valve mask (e.g., an artificial manual breathing unit (AMBU) bag) is attached to the tracheal tube for subsequent manual ventilation (manual resuscitation).

Use of manual resuscitators (e.g., AMBU bags) to ventilate patients is called “bagging” and is often necessary in critical care situations when the patient's breathing is insufficient (e.g., respiratory failure) or has ceased completely (e.g., respiratory arrest). During these critical care situations, manual resuscitation with AMBU bags (i.e., a manual bag valve mask) are preferably used instead of mechanical ventilators because the use of manual resuscitators allow emergency care providers a greater degree of freedom to perform various medical procedures and a greater degree of maneuverability for both the patient and care provider when performing these procedures.

Although in theory the tracheal tube (e.g., an endotracheal tube) will always remain unobstructed during these critical care situations, various bodily fluids and secretions often enter the tracheal tube, thus potentially blocking the patient's airway and complicating any procedure being performed during these critical care situations. For example, it is not uncommon for blood, mucus (e.g., mucus plugs), and other bodily fluids to enter and at least partially obstruct the tracheal tube during these critical care situations. To prevent, aspiration and further complications of any medical procedure being performed during these critical care situations, blood, mucus (e.g., mucus plugs), or any other bodily fluids obstructing the tracheal tube must be removed, preferably as quickly as possible.

When removing blood, mucus (e.g., mucus plugs), or any other bodily fluids from the tracheal tube, it is conventionally well accepted that the AMBU bag (i.e., a manual bag valve mask) must be detached from the tracheal tube. After detaching the AMBU bag from the tracheal tube, a suction device, having a smaller diameter than the tracheal tube, must be fed into the tracheal tube and suction of any object (e.g., blood, mucus, etc.) at least partially obstructing the tracheal tube must occur to clear the tube.

Numerous problems exist with this conventional process of removing the AMBU bag from the tracheal tube when suctioning any obstructive object within the tube. For example, sterility of the AMBU bag becomes compromised especially if the same AMBU bag is reattached to the patient's tracheal tube and manual resuscitation is resumed with this same AMBU bag. Thus, the patient may be at increased risk for various bacterial or viral infections (e.g., a nosocomial infection). In addition, the medical care provider performing a particular medical procedure is at risk of being exposed to the patient's bodily fluids (e.g., blood, mucus, etc.) and blood borne pathogens while detaching the AMBU bag in order to suction the tracheal tube as needed. Also, overall medical procedure time is increased during these critical care situations due to the required, conventional process of detaching and reattaching the AMBU bag when suctioning is necessary.

### BRIEF SUMMARY OF THE INVENTION

Therefore, it is an object of the devices and methods disclosed herein to reduce or prevent the problems associated with manual resuscitation by providing a device having dual manual resuscitation and suction capabilities. In certain aspects, these devices will advantageously reduce overall medical procedure time, reduce a patient's potential risk for bacterial and/or viral infection, and reduce the medical provider's potential risk of being exposed to a patient's bodily fluids by providing a closed, sterile system configured to have dual manual resuscitation and suction capabilities.

Disclosed is a valve assembly for a resuscitator including a valve body defining a gas passageway therethrough, the valve body including a first inlet for attaching a compressible bag, a second inlet for attaching a suction device, and a first outlet for attaching an endotracheal tube, wherein the second inlet and the first outlet are axially aligned, and the first inlet is axially perpendicular to both the second inlet and the first outlet; and a valve positioned within the valve body axially aligned between the second inlet and the first outlet, the valve opening in the direction of the first outlet. In certain aspects, the valve is a check valve, and the suction device includes a tube arranged to be advanced through the check valve and into the endotracheal tube such that the endotracheal tube can be suctioned without having to detach the endotracheal tube from the valve body.

In certain aspects, the valve assembly further includes an injection port positioned laterally adjacent to and in fluid communication with the first outlet. The injection port may be an elongate tubular member preferably positioned between the first outlet and the valve on an outer surface of the valve assembly. In certain aspects, the injection port is positioned on an outer peripheral surface of the valve assembly below the check valve.

Also disclosed is a resuscitator including a valve assembly having a check valve arranged within a valve body, the valve body defining a gas passageway therethrough, the valve body including a first inlet, a second inlet and a first outlet, wherein the second inlet and the first outlet are axially aligned, and the first inlet is axially perpendicular to both the second inlet and the first outlet, and wherein the valve is arranged within the valve body between the second inlet and the first outlet in axial alignment with the second inlet and the first outlet; a compressible bag in fluid communication with the first inlet; a suction device removably attached to the second inlet; and an endotracheal tube removably attached to the first outlet. The suction device includes a tube arranged to be advanced through the valve and into the endotracheal tube such that the endotracheal tube can be suctioned without having to detach the endotracheal tube from the valve body. The disclosed resuscitator is a closed, sterile system configured for dual purposes, namely manual resuscitation and suction.

In certain aspects, the resuscitator further includes a second outlet that is in fluid communication with a positive end expiratory pressure valve. In certain aspects, the positive end expiratory pressure valve is co-planar relative to the first inlet and perpendicular relative to both the second inlet and first outlet.

In certain aspects, at least one of the second inlet and the first outlet of the resuscitator defines an annular collar and recess for attaching a cylindrical fitting of at least one of the suction device and the endotracheal tube.

In certain aspects, the resuscitator further includes an injection port positioned laterally adjacent to and in fluid communication with the first outlet.

In certain aspects, the injection port is an elongate tubular member positioned between the first outlet and the valve on an outer surface of the valve assembly.

In certain aspects, the suction device of the resuscitator is attached to the second inlet via a threaded engagement.

In certain aspects, the suction device of the resuscitator is attached to the second inlet via a friction fit.

In certain aspects, the endotracheal tube of the resuscitator is removably attached to the first outlet via a friction fit.

In certain aspects, the endotracheal tube of the resuscitator is removably attached to the first outlet via a threaded engagement.

In certain aspects, the compressible bag of the resuscitator is removably attached to the first inlet.

In certain aspects, the compressible bag of the resuscitator is removably attached to the first inlet via a friction fit.

In certain aspects, the compressible bag of the resuscitator is removably attached to the first inlet via a threaded engagement.

Additional features, aspects and advantages of the invention will be set forth in the detailed description which follows, and in part will be readily apparent to those skilled in the art from that description or recognized by practicing the invention as described herein. It is to be understood that both the foregoing general description and the following detailed description present various embodiments of the invention, and are intended to provide an overview or framework for understanding the nature and character of the invention as it is claimed. The accompanying drawings are included to provide a further understanding of the invention, and are incorporated in and constitute a part of this specification.

### BRIEF DESCRIPTION OF THE DRAWINGS

These and other features, aspects and advantages of the present invention are better understood when the following detailed description of the invention is read with reference to the accompanying drawings, in which:

FIG. 1 is a perspective view of the resuscitator according to the invention;

FIGS. 2A-2C show exemplary views of the resuscitator in use; for example, FIG. 2A shows the suction device in a retracted state in which the suction tube has not been advanced through the check valve; FIG. 2B shows the suction tube being advanced through the check valve and entering into the endotracheal tube; FIG. 2C shows suction tube being advanced through the endotracheal tube;

FIGS. 3A and 3B show various magnified views of the valve assembly; for example, FIG. 3A shows the valve assembly including the check valve in which the suction tube has not been advanced through the check valve; FIG. 3B shows the valve assembly in which the suction tube has been advanced through the check valve;

FIG. 4 is an exploded view of the resuscitator; and

FIG. 5 is an exploded view of a second embodiment of the resuscitator according to the invention.

### DETAILED DESCRIPTION OF THE INVENTION

The present invention will now be described more fully hereinafter with reference to the accompanying drawings in which exemplary embodiments of the invention are shown. However, the invention may be embodied in many different forms and should not be construed as limited to the representative embodiments set forth herein. The exemplary embodiments are provided so that this disclosure will be both thorough and complete, and will fully convey the scope of the invention and enable one of ordinary skill in the art to make, use and practice the invention. Like reference numbers refer to like elements throughout the various drawings.

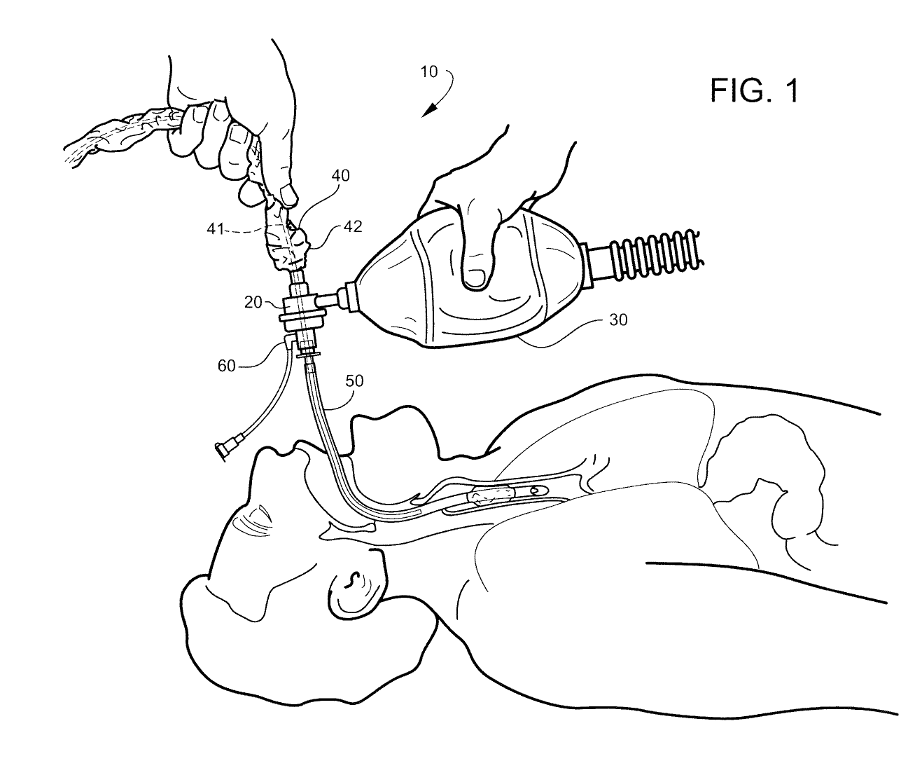

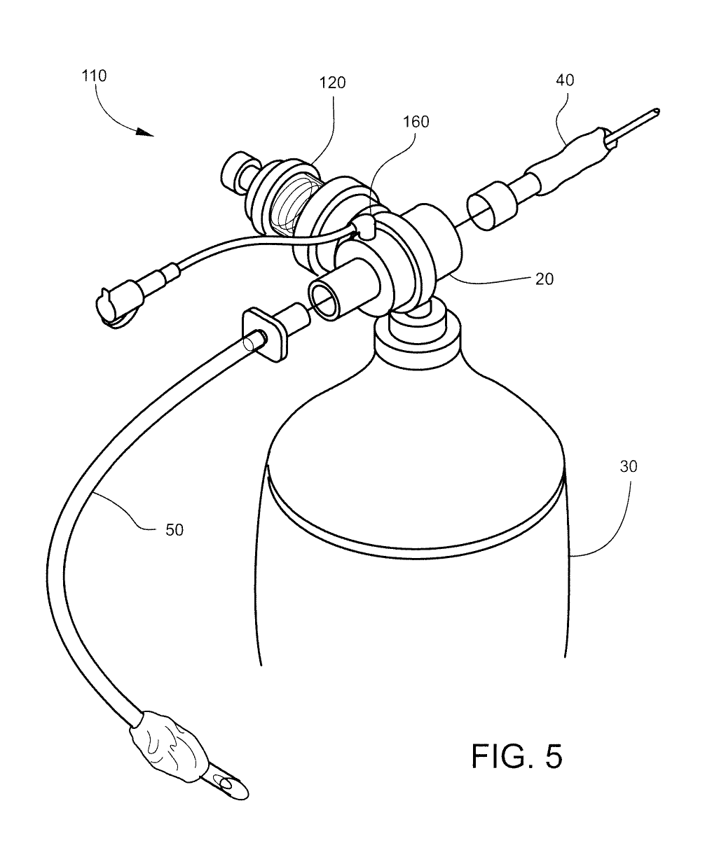

Disclosed is resuscitator 10, 110 having dual manual resuscitation and suction capabilities. FIGS. 1 and 5 show two embodiments of the resuscitator 10, 110. FIG. 1 shows an exemplary embodiment of a resuscitator 10 that includes a valve assembly 20, a compressible bag 30, and a suction device 40. In certain aspects, the valve assembly 20 is attached to or configured to attach to an endotracheal tube 50. In certain aspects, resuscitator 10, 110 having dual manual resuscitation and suction capabilities advantageously reduces overall medical procedure time, reduces a patient's potential risk for bacterial or viral infection, and reduces the medical providers potential risk of being exposed to a patient's bodily fluids.

The valve assembly 20 of resuscitator 10, 110 includes a valve body defining a gas passageway therethrough. The valve assembly (e.g., the valve body or housing) is preferably made from a rigid material such as a molded thermoplastic resin (e.g., injection molded or blow molded) that includes, but is not limited to, polypropylene, polyethylene, polyester, polystyrene, polyvinyl chloride, or any combination thereof. In certain aspects, the valve body is a single, unitary body formed from the molded thermoplastic resin. In certain aspects, the valve body may include a plurality of components configured to attach to one another to form the valve body.

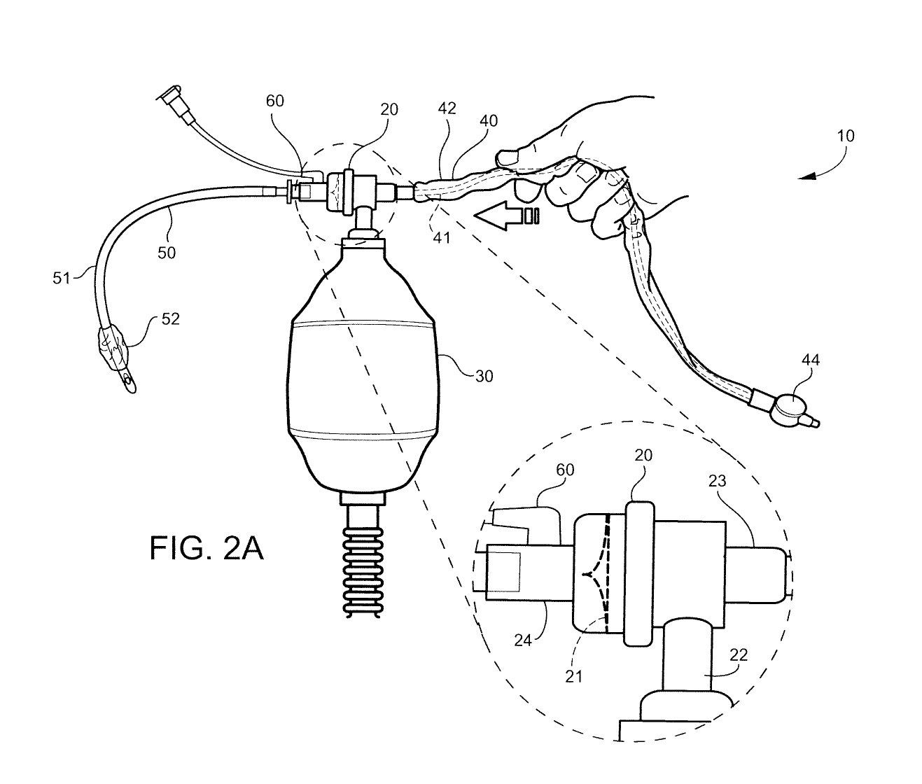

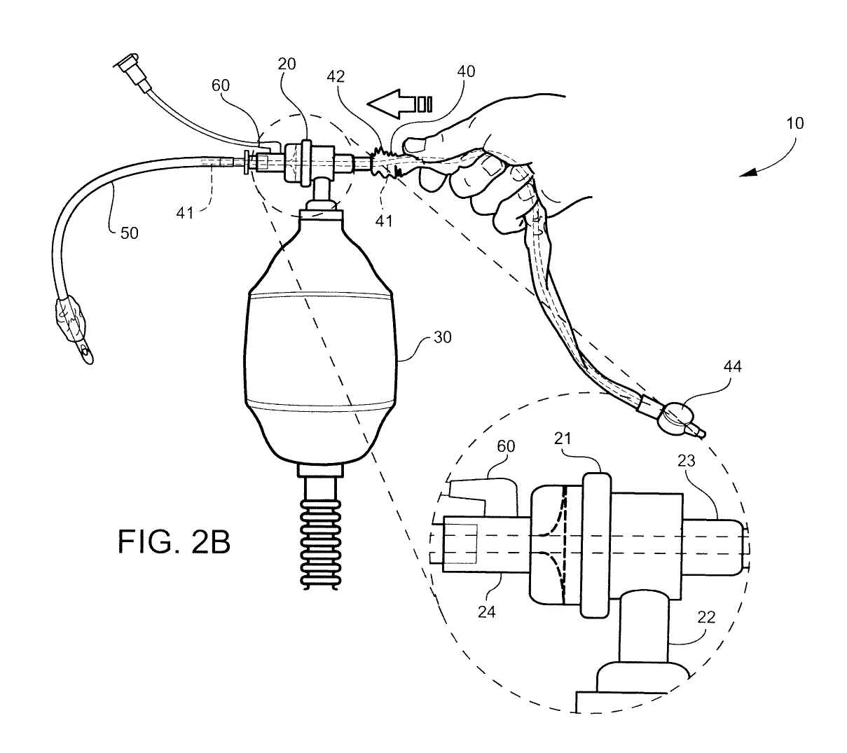

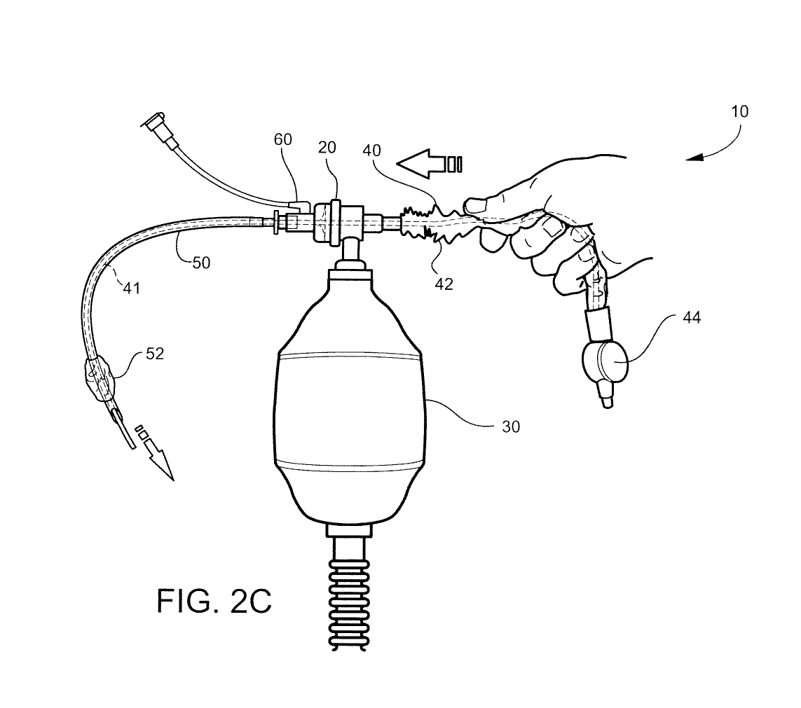

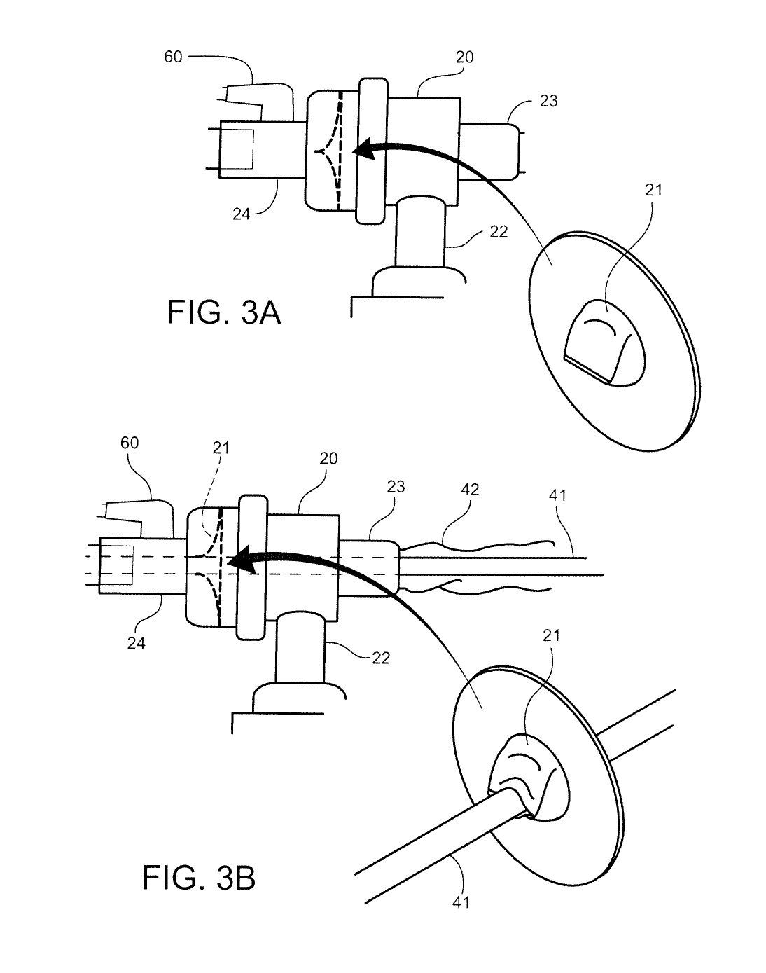

As shown, for example, in FIGS. 2A-2C, 3A, and 3B, the valve body includes a check valve 21, a first inlet 22, a second inlet 23, and a first outlet 24. In this aspect, the second inlet 23 and the first outlet 24 are axially aligned, and the first inlet 22 is axially perpendicular to both the second inlet 23 and the first outlet 24. In certain aspects, the first inlet 22 is also offset relative to the second inlet 23 and the first outlet 24. In certain aspects, the check valve 21 is arranged within the valve body between the second inlet 23 and the first outlet 24 in axial alignment with the second inlet 23 and the first outlet 24. The check valve 21 is preferably made of a flexible, elastomeric material having resiliency and/or memory. In certain aspects, this flexible, elastomeric material is biocompatible, non-immunogenic, or a combination thereof. This flexible, elastomeric may include polypropylene, polyvinyl chloride, a synthetic rubber, a natural rubber, or any combination thereof. As shown in FIG. 2B, the flexible, elastomeric material allows the suction tube 41 to be advanced through the check valve 21 but is resilient such that no permanent deformation occurs when the suction tube 41 is either advanced through or retracted from the check valve 21.

The compressible bag 30 is attached to the valve assembly 20 and is in fluid communication with the first inlet 22. In certain aspects, the compressible bag 30 may be permanently attached to the valve assembly 20. However, in other aspects, the compressible bag 30 may be detachable from the valve assembly 20. For example, when the compressible bag 30 is detachable from the valve assembly 20, the valve assembly 20 and the compressible bag 30 will include portions configured to mate to one another. These portions may include snap-fit attachments or a female thread portion and a male thread portion allowing for attachment of compressible bag 30 to the valve assembly 20. The compressible bag 30 may include, or be similar to, a compressible bag within a manual resuscitator manufactured by SOLMED™, a compressible bag within a manual resuscitator manufactured by AMBU®, or a compressible bag within the PORTEX® manual resuscitator.

In certain aspects, the suction device 40 is attached to the second inlet 23 of the valve assembly 20. For example, in certain aspects, the suction device is permanently attached to the second inlet 23 of the valve assembly 20. In other aspects, the suction device 40 is removably attached to the second inlet 23 of the valve assembly 20. For example, when the suction device 40 is removably attached to the second inlet 23 of the valve assembly 20, the valve assembly and suction device will include portions configured to mate to one another. For example, these portions may include snap-fit attachments or a female thread portion and a male thread portion allowing for attachment of suction device to the valve assembly.

In certain aspects, the suction device 40 includes a suction tube 41 placed inside and completely surrounded by a flexible polymeric sheath 42 (e.g., a plastic sheath). This suction tube 41/sheath 42 configuration preferably creates a closed system that is hygienic and reduces or prevents loss of the suction tube's sterility during use of the resuscitators 10, 110. The suction device 40 further preferably includes either a mechanical device 44 (e.g., a “thumb control” device) or an opening (not shown) for selectively creating and controlling a vacuum within the suction tube when suctioning is necessary. The suction device 40 disclosed herein may include, for example, an endotracheal suction catheter such as the Kimberly Clark® Trach Care Closed System Catheter, 14 Fr. Model 2210, Model 2155, Model 22703, or Model 22715.

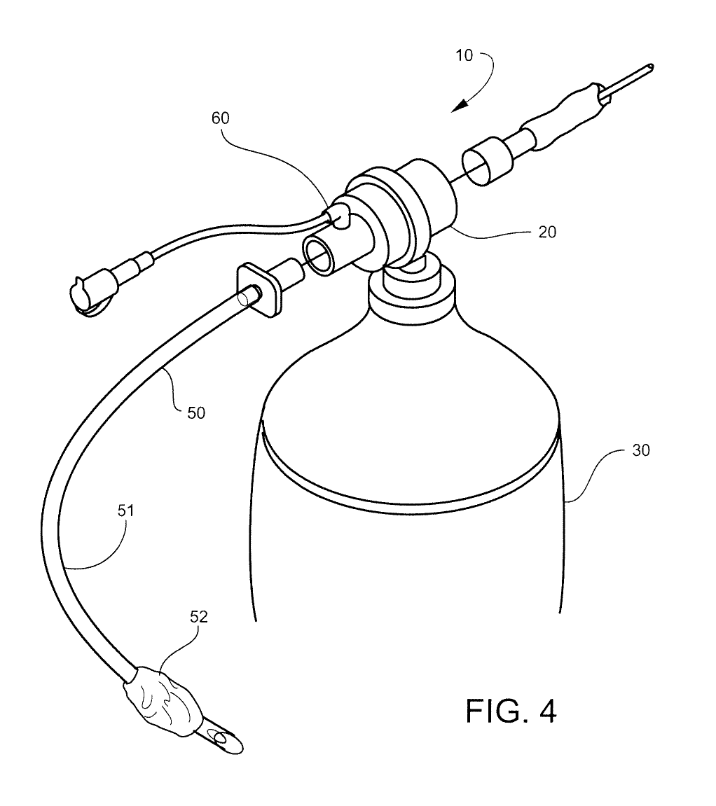

In certain aspects, an endotracheal tube 50 is removably attached to the first outlet 24 of the valve assembly 20. For example, in certain aspects, the first outlet 24 and endotracheal tube 50 may be configured to friction fit (e.g., interference fit) to one another or to snap-fit to one another. In certain aspects and as depicted in FIGS. 2A, 2C, and 4, the endotracheal tube 50 includes an elongate, non-expandable tube 51 and an inflatable bladder 52 directly attached on a portion of the elongate, non-expandable tube. In certain aspects, the elongate, non-expandable tube 51 may has a diameter ranging from 2.0 mm to 10.0 mm, 3.0 mm to 9.5 mm, 4.5 mm to 8.5 mm, and 5.0 mm to 7.0 mm, and the portion the endotracheal tube having the inflatable bladder 52 directly attached on a portion of the elongate, non-expandable tube may inflate to have an overall diameter ranging from 3.0 mm to 13.0 mm, 4.5 mm to 12.0 mm, 6.0 mm to 10.0 mm, and 6.5 mm to 8.0 mm. In certain aspects, the suction tube 41 has a diameter ranging from 1.0 mm to 5.0 mm, 1.5 mm to 4.5 mm, 2.0 mm to 4.0 mm, 2.5 mm to 3.5 mm, 3.0 mm to 3.5 mm.

The valve assembly 20 may include a port 60, 160 used for administering, for example, saline (i.e., sterile saline) or a desired medicament (e.g., salbutamol, atropine, epinephrine, ipratropium, and lidocaine). As shown, for example, in FIGS. 1, 3A, and 3B, the port 60 may be positioned beneath the check valve 21 such that saline or the desired medicament never contacts the check valve 21 if administration of saline and/or any desired medicament is necessary. However, as shown in FIG. 5, in certain aspects the port 160 may also be positioned above the check valve 21. In certain aspects, port 60, 160 is an injection port positioned laterally adjacent to and in fluid communication with the first outlet. The injection port may be an elongate tubular member preferably positioned between the first outlet and the valve on an outer surface of the valve assembly. In certain aspects, the injection port is positioned on an outer peripheral surface of the valve assembly below the check valve. While in other aspects, the injection port may be positioned above the check valve.

The valve assembly 20 and resuscitators 10, 110 disclosed herein are arranged such that a suction tube 41 can be advanced through the check valve 21 and into the endotracheal tube 50 such that the endotracheal tube can be suctioned without having to detach the endotracheal tube from the valve body.

In certain aspects and as depicted in FIG. 5, the valve assembly 20 may further include a positive end expiratory pressure valve (i.e., a PEEP valve) 120. The PEEP valve 120 may be either permanently attached or removably attached to the valve assembly 20. For example, when the PEEP valve 120 is configured to be removably attached of the valve assembly 20, the valve assembly and PEEP valve will include portions configured to mate to one another. For example, these portions may include snap-fit attachments or a female thread portion and a male thread portion allowing for attachment of the PEEP valve to the valve assembly. In certain aspects, it may be advantageous to include the PEEP valve to avoid or reduce the risk of end-expiratory alveolar collapse during respiration.

FIG. 1 shows a perspective view during use of the resuscitator according to the invention. Specifically, FIG. 1 shows an intubated subject having the resuscitator 10 attached to the endotracheal tube 50. As further shown in FIG. 1, the suction tube 41 has been advanced through the check valve into the endotracheal tube in order to suction (i.e., clear) any object (e.g., blood, mucus, etc.) partially or completely occluding the endotracheal tube. FIG. 2A depicts the suction device in a retracted state in which the suction tube has not been advanced through the check valve. FIG. 2B further depicts the suction tube being advanced through the check valve and entering into the endotracheal tube.

The valve assembly 20, compressible bag 30, and suction device 40 may be packaged separately and assembled accordingly when needed during medical treatment. Alternatively, valve assembly 20, compressible bag 30, and suction device 40 may be packaged as a resuscitator kit. For example, within such a resuscitator kit, resuscitator 10, 110 may already be pre-assembled, just requiring the medical professional to remove the resuscitator from the packaging in order to use. Alternatively, resuscitator 10, 110 may be partially assembled within the package requiring the user to assemble the components within the kit as needed.

The foregoing description provides embodiments of the invention by way of example only. It is envisioned that other embodiments may perform similar functions and/or achieve similar results. Any and all such equivalent embodiments and examples are within the scope of the present invention and are intended to be covered by the appended claims.

What is claimed is:

1. A valve assembly for a resuscitator, comprising:
a valve body defining a gas passageway therethrough, the valve body including a first inlet for attaching a compressible bag, a second inlet for attaching a suction device, and a first outlet for attaching an endotracheal tube, wherein the second inlet and the first outlet are axially aligned, and the first inlet is axially perpendicular to both the second inlet and the first outlet; and
a valve positioned within the valve body axially aligned between the second inlet and the first outlet, the valve opening in the direction of the first outlet.

2. The valve assembly of claim 1, wherein the valve is a check valve, and wherein the suction device includes a tube arranged to be advanced through the check valve and into the endotracheal tube such that the endotracheal tube can be suctioned without having to detach the endotracheal tube from the valve body.

3. The valve assembly of claim 1, wherein the valve body further comprises a second outlet in fluid communication with a positive end expiratory pressure valve.

4. The valve assembly of claim 1, wherein at least one of the second inlet and the first outlet defines an annular collar and recess for attaching a cylindrical fitting of at least one of the suction device and the endotracheal tube.

5. The valve assembly of claim 1, further comprising an injection port positioned laterally adjacent to and in fluid communication with the first outlet.

6. The valve assembly of claim 5, wherein the injection port is an elongate tubular member positioned between the first outlet and the valve on an outer surface of the valve assembly.

7. A resuscitator, comprising:
a valve assembly comprising a check valve arranged within a valve body, the valve body defining a gas passageway therethrough, the valve body including a first inlet, a second inlet and a first outlet, wherein the second inlet and the first outlet are axially aligned, and the first inlet is axially perpendicular to both the second inlet and the first outlet, and wherein the valve is arranged within the valve body between the second inlet and the first outlet in axial alignment with the second inlet and the first outlet;
a compressible bag in fluid communication with the first inlet;
a suction device removably attached to the second inlet; and
an endotracheal tube removably attached to the first outlet; wherein:
the suction device includes a tube arranged to be advanced through the valve and into the endotracheal tube such that the endotracheal tube can be suctioned without having to detach the endotracheal tube from the valve body.

8. The resuscitator of claim 7, wherein the resuscitator further comprises a second outlet that is in fluid communication with a positive end expiratory pressure valve.

9. The resuscitator of claim 7, wherein at least one of the second inlet and the first outlet defines an annular collar and recess for attaching a cylindrical fitting of at least one of the suction device and the endotracheal tube.

10. The resuscitator of claim 7, further comprising an injection port positioned laterally adjacent to and in fluid communication with the first outlet.

11. The resuscitator of claim 10, wherein the injection port is an elongate tubular member positioned between the first outlet and the valve on an outer surface of the valve assembly.

12. The resuscitator of claim 7, wherein the suction device is attached to the second inlet via a threaded engagement.

13. The resuscitator of claim 7, wherein the suction device is attached to the second inlet via a friction fit.

14. The resuscitator of claim 7, wherein the endotracheal tube is removably attached to the first outlet via a friction fit.

15. The resuscitator of claim 7, wherein the endotracheal tube is removably attached to the first outlet via a threaded engagement.

16. The resuscitator of claim 7, wherein the compressible bag is removably attached to the first inlet.

17. The resuscitator of claim 16, wherein the compressible bag is removably attached to the first inlet via a friction fit.

18. The resuscitator of claim 16, wherein the compressible bag is removably attached to the first inlet via a threaded engagement.
